(19) United States
(12) Patent Application Publication
(10) Pub. No.: US 2004/0068215 A1
(43) Pub. Date: Apr. 8, 2004

(54) Osteoarthritis knee brace apparatus and method

(76) Inventors: Jeremy Adelson, Bainbridge Island, WA (US); Stewart L. Atkinson, Bainbridge Island, WA (US); James G. Cairns JR., Bainbridge Island, WA (US)

Correspondence Address: Gerald L. Fellows, Michael Best & Friedrich LLP, 100 East Wisconsin Avenue, Milwaukee, WI, 53202-4108, US

(21) Appl. No.: 10/266,394

(22) Filed: Oct. 8, 2002

(51) Int. Cl. A61F 5/00

(52) U.S. Cl. 602/26

(57) ABSTRACT

A knee brace for coupling to a patient's knee. The knee brace can include a flexion/extension joint, a first arm coupled to the flexion/extension joint, and a first ramp positioned to engage the first arm. The first ramp can be shaped to move the first arm towards the patient's leg as the patient's knee extends and away from the patient's leg as the patient's knee flexes. Alternatively, the first ramp can be shaped to move the first arm away from the patient's leg as the patient's knee extends and toward the patient's leg as the patient's knee flexes.

### FIELD OF THE INVENTION

This invention relates generally to knee braces, and more particularly to an apparatus and method for a knee brace to treat osteoarthritis.

### BACKGROUND OF THE INVENTION

A healthy knee joint includes a symmetrical space between the femur and the tibia and an even distribution of pressure in the medial and lateral knee compartments. The space between the femur and the tibia is normally about one-quarter of an inch. The space between the femur and the tibia decreases when uni-compartmental osteoarthritis occurs in the patient's knee. Uni-compartmental osteoarthritis can occur in a patient's knee after an injury or as a result of aging. If left untreated, uni-compartmental osteoarthritis can progress until the space between the femur and the tibia is eliminated and the femur contacts the tibia, resulting in erosion of the tibia. In addition, uni-compartmental osteoarthritis can also change the normal angle between the femur and the tibia. This change in angle between the femur and the tibia can lead to a varus condition (i.e., the patient becomes bow-legged) or a valgus condition (i.e., the patient becomes knock-kneed).

Uni-compartmental arthritis of the knee has conventionally been treated in the following three ways: (1) anti-inflammatory and analgesic treatments; (2) high tibial osteotomy; and (3) total knee arthroplasty or knee replacement. Anti-inflammatory and analgesic treatments only attempt to reduce the swelling of the knee joint, rather than treat the cause of the osteoarthritis. High tibial osteotomy is a surgical procedure that involves the removal of a triangular segment of the tibia to correct the change of angle between the femur and the tibia. Total knee arthroplasty or knee replacement is a surgical procedure that is generally the last option for a patient suffering from uni-compartmental osteoarthritis.

Conventional knee braces have been used in an attempt to temporarily reduce the pressure on the knee compartment with osteoarthritis, to prolong the time until the patient must have a surgical procedure, or to prevent the patent from requiring a surgical procedure. Generally, these knee braces have attempted to apply a three-point force or an unloading force to the patient's knee. This three-point force often consists of three separate forces applied to the patient's leg—a first force generally directed above the patient's knee, a second force generally directed toward or through the patient's knee, and a third force generally directed below the patient's knee. The first and third forces are applied in the same direction, while the second force is applied in the opposite direction to the first and third forces. Conventional knee braces apply these three forces throughout the patient's gait, i.e., continuously throughout flexion and extension. However, the unloading force is often most effective and most necessary only at full extension. If the force is also applied throughout flexion, the knee brace can be uncomfortable for the patient to wear.

In light of the problems and limitations described above, a need exists for a method and apparatus for a knee brace that applies one or more forces to a patient's knee in order to relieve uni-compartmental osteoarthritis. A need also exists for a knee brace that applies a greater unloading force during extension than during flexion to make the knee brace more comfortable for the patient to wear. A need also exists for a knee brace including a pair of arms and a joint between the pair of arms that can either increase or decrease a distance between the arms and the patient's leg during flexion or extension. A need still further exists for a knee brace including one or more arms and one or more adjustable ramps that move the arms medially and laterally with respect to the patient's leg as the patient's knee flexes and extends. A need still further exists for a knee brace with low-profile, yet high-strength components capable of exerting unloading forces on a patient's knee. Finally, a need exists for a knee brace that is comprised of inexpensive components, that is simple to manufacture, that is easy to assembly, that is easy to customize for a particular patient, and that is easy to repair. Each embodiment of the present invention achieves one or more of these results.

### SUMMARY OF THE INVENTION

Some embodiments of the invention provide a knee brace for coupling to a patient's knee, including a flexion/extension joint, a first arm coupled to the flexion/extension joint, and a first ramp positioned to engage the first arm. The first ramp can be shaped to move the first arm towards the patient's leg as the patient's knee extends and away from the patient's leg as the patient's knee flexes. Alternatively, the first ramp can be shaped to move the first arm away from the patient's leg as the patient's knee flexes and toward the patient's leg as the patient's knee extends. In some embodiments, the first ramp includes a contoured surface shaped to move the first arm gradually. The first ramp can include a ramp cage or cage member with a first aperture through which the first arm is positioned, and the first aperture of the cage member can define the contoured surface within which the first arm moves. In other embodiments, the first ramp includes a single plate along which the first arm moves.

The knee brace can include a pair of arms for coupling to the patient's knee and a joint between the arms. The joint can allow flexion and extension of the patient's knee, while a medial/lateral distance between at least one arm of the pair of arms and the patient's leg is greater during flexion than during extension, or, alternatively, greater during extension than during flexion. The joint can also be positioned with respect to the patient's knee and configured so that at least one arm of the pair of arms moves laterally as the patient's knee flexes and medially as the patient's knee extends, or, alternatively, medially as the patient's knee flexes and laterally as the patient's knee extends.

Some embodiments of the invention provide a knee brace including a pair of arms for coupling to the patient's knee, a first joint coupled to the pair of arms to allow flexion and extension of the patient's knee, and a second joint in the brace for allowing controlled medial and lateral inclination relative to the first joint of one or more arms of the pair of arms. The medial/lateral inclination of the one or more arms can be more lateral during flexion and more medial during extension. Alternatively, the medial/lateral inclination of the one or more arms can be more medial during flexion and more lateral during extension.

A knee brace according to the invention can also include a pair of arms for coupling to the patient's knee and means coupled to the pair of arms for allowing flexion and extension of the patient's knee and for increasing a medial/lateral distance between one or more arms of the pair of arms and the patient's knee during flexion and decreasing the medial/lateral distance during extension. Alternatively, the means coupled to the pair of arms can decrease a medial/lateral distance between one or more arms of the pair of arms and the patient's knee during flexion and increase the medial/lateral distance during extension.

In some embodiments, the knee brace includes a first shell substantially conforming to the patient's thigh, a second shell substantially conforming to the patient's calf, a first arm coupled to the first shell, a second arm coupled to the second shell, and a flexion/extension joint coupled between the first arm and the second arm. In addition, the knee brace can include a first ramp positioned to engage the first arm and a second ramp positioned to engage the second arm. The first and second ramps can be shaped to move the first and second arms toward the patient's leg as the patient's knee extends and away from the patient's leg as the patient's knee flexes. Alternatively, the first and second ramps can be shaped to move the first and second arms away from the patient's leg as the patient's knee extends and toward the patient's leg as the patient's knee flexes.

According to a method of the invention, a brace is secured to the patient's knee and the patient's knee is allowed to flex and extend. A medial/lateral force can be exerted on the patient's leg as the patient's knee flexes and extends. The medial/lateral force can be exerted on at least one of the following points: a first point generally above the patient's knee, a second point generally at the patient's knee, and a third point generally below the patient's knee. In addition, the medial/lateral force can be increased as the patient's knee extends and decreased as the patient's knee flexes.

According to another method of the invention, a first force is directed toward a first point generally above the patient's knee, a second force is directed toward a second point generally below the patient's knee, and a third force is directed generally away from the patient's knee. Also, the first and second forces toward the patient's leg are increased as the patient's knee extends and decreased as the patient's knee flexes. Alternatively, a first force is directed away from a first point generally above the patient's knee, a second force is directed away from a second point generally below the patient's knee, and a third force is directed generally towards the patient's knee. Also, the first and second forces away from the patient's leg are increased as the patient's knee extends and decreased as the patient's knee flexes.

### BRIEF DESCRIPTION OF THE DRAWINGS

The present invention is further described with reference to the accompanying drawings which show preferred embodiments of the present invention. Although not specifically described herein, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a perspective view of a knee brace embodying the invention;

FIG. 2 is a perspective view of a hinge assembly (including a cover) for use with the knee brace of FIG. 1;

FIG. 3 is a perspective view of the hinge assembly of FIG. 2 with the cover removed;

FIG. 4 is a lateral side elevational view of the hinge assembly of FIG. 3;

FIG. 5 is an anterior side elevational view of the hinge assembly of FIG. 3;

FIG. 6 is a posterior side elevational view of the hinge assembly of FIG. 3;

FIG. 7 is a medial side elevational view of the hinge assembly of FIG. 3;

FIG. 8 is an exploded perspective view of the hinge assembly of FIG. 2 including the cover;

FIG. 9 is a side elevational view of an alternative embodiment of the hinge assembly embodying the invention;

FIG. 10A is a perspective view of another alternative embodiment of the hinge assembly embodying the invention, and FIGS. 10B-10D are perspective views of inserts for use with the hinge assembly of FIG. 10A;

FIG. 11 is a posterior side elevational view of another alternative embodiment of the hinge assembly embodying the invention;

FIG. 12A is a perspective view of another alternative embodiment of the hinge assembly embodying the invention, and FIGS. 12B-12E are front elevational views of inserts for use with the hinge assembly of FIG. 12A;

FIG. 13A is a perspective view of another alternative embodiment of the hinge assembly embodying the invention, FIG. 13B is a front elevational view of a wedge for use with the hinge assembly of FIG. 13A, and FIGS. 13C-13F are side elevational views of wedges for use with the hinge assembly of FIG. 13A;

FIG. 14 is a perspective view of another alternative embodiment of a knee brace embodying the invention;

FIG. 15 is a lateral side elevational view of a hinge assembly for use with the knee brace of FIG. 14;

FIG. 16 is an exploded perspective view of an upper shell, a cylinder joint and an upper arm for use with the knee brace of FIG. 14;

FIG. 17 is an exploded perspective view of the cylinder joint and the upper arm of FIG. 16;

FIG. 18 is an anterior side elevational view of the knee brace of FIG. 14 with the cylinder joint in a first position;

FIG. 19 is an anterior side elevational view of the knee brace of FIG. 14 with the cylinder joint in a second position;

FIG. 20 is a perspective view of an upper shell for use with another alternative embodiment of a knee brace embodying the invention; and

FIGS. 21A-21D are front elevation views of the hinge assembly of FIG. 2 attached at different positions with respect to a patient's leg.

### DETAILED DESCRIPTION OF THE PREFERRED EMBODIMENTS

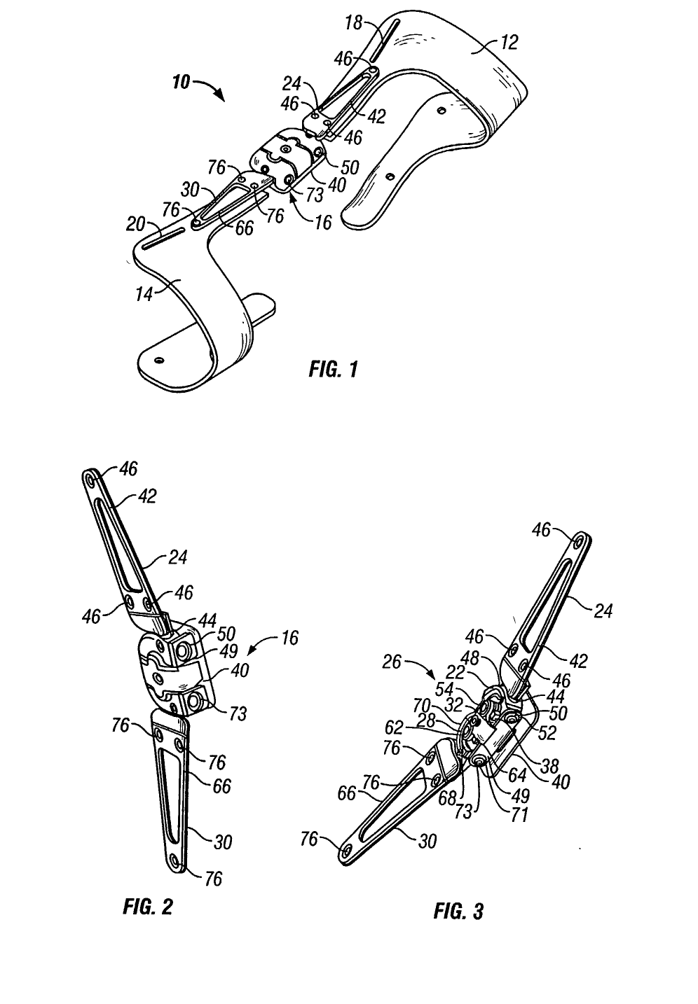

FIG. 1 illustrates one embodiment of a knee brace 10 according to the present invention. The knee brace 10 includes an upper shell 12 coupled to a lower shell 14 by a joint in the form of a hinge assembly 16. Although the joint is shown in this embodiment as a hinge assembly, the joint can be any suitable structure used to join the shells 12 and 14 (e.g., a hinge, a universal joint, or a ball-and-socket joint). The upper shell 12 and the lower shell 14 can be custom made according to the shape of each individual patient's leg, or the upper shell 12 and the lower shell 14 can be off-the-shelf-components. The upper shell 12 and the lower shell 14 can be constructed of composites, reinforced composites (such as carbon-fiber composites or fiberglass-reinforced thermoplastics), fiberglass, plastic, metals, metal alloys, hardened polymers, or any other suitable material. The upper shell 12 can substantially conform to the patient's thigh and the lower shell 14 can substantially conform to the patient's calf. As shown in FIG. 1, the shells include two substantially vertical members and one substantially horizontal member. However, the shells 12 and 14 can have any suitable shape or configuration. For example, the vertical member opposite to the hinge assembly 16 can be omitted and/or the horizontal member can be extended to wrap around a greater portion of the patient's leg. In some embodiments, the upper shell 12 and the lower shell 14 can be secured to the patient's right or left leg with two or more straps (not shown) positionable through an upper slot 18 and a lower slot 20. In other embodiments, loops (e.g., molded plastic loops) can be riveted to the upper shell 12 and the lower shell 14 and straps can be secured through the loops. Also, a combination of slots in the shells and loops secured to the shells can be used to attach straps to the brace. In addition, the shells 12 and 14 can be secured to the patient's leg in other suitable manners, such as with straps or other suitable components that wrap around the patient's leg and are releasably fastened with buckles, clips, mating pins and apertures, snap-fit connections, press-fit connections, hook-and-loop fasteners, etc.

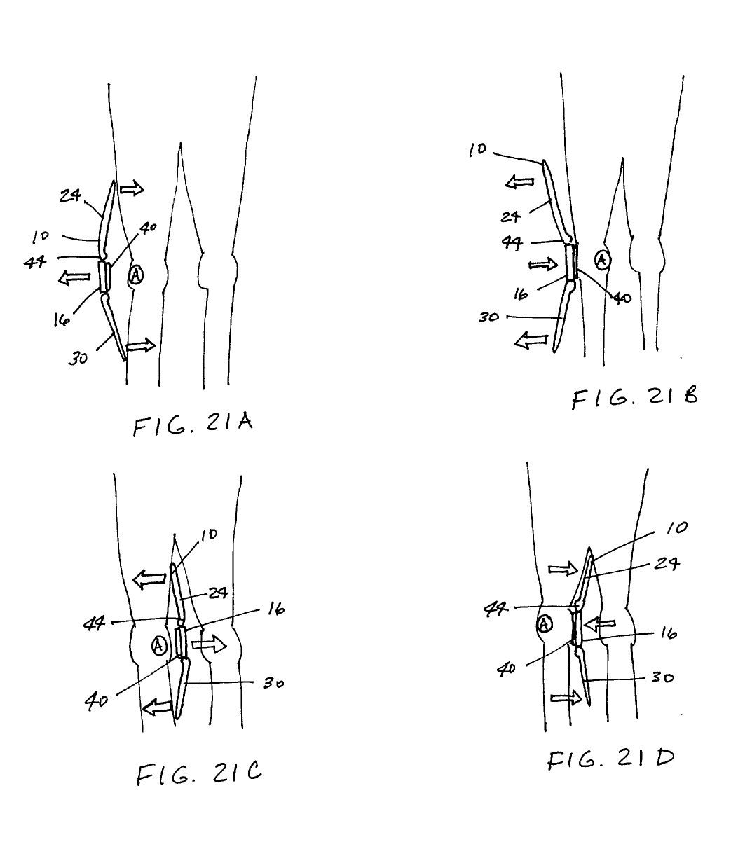

The knee brace 10 can be constructed for either the patient's left or right leg. The side of the knee brace 10 that includes the hinge assembly 16 can be positioned on either side of either one of the patient's knees. For example, the hinge assembly 16 can be positioned on a medial or lateral side of either one of the patient's knees with respect to a knee compartment having osteoarthritis. As used herein and in the appended claims, the term “medial” means toward an imaginary midline dividing the body into equal right and left halves. Conversely, the term “lateral” as used herein and in the appended claims means away from this imaginary midline. For example, as shown in FIGS. 21A and 21B, the hinge assembly 16 is positioned on a lateral side of the patient's knee, and, as shown in FIGS. 21C and 21D, the hinge assembly 16 is positioned on a medial side of the patient's knee. A knee compartment having arthritis is designated by the letter “A” in FIGS. 21A-21D. Other configurations for the knee brace 10 with respect to the location of the arthritic knee compartment are also possible. In addition, the schematic representations in FIGS. 21A-21D have been exaggerated to more clearly illustrate possible configurations of the knee brace 10 with respect to an arthritic knee compartment. FIGS. 21A-21D generally illustrate the application of a three-point force (in the form of three separate forces) on the patient's leg in order to relieve or unload the arthritic compartment. For the three-point force, one medial/lateral force can be directed toward or away from the patient's knee and two medial/lateral forces can be directed in the opposite direction generally above and below the patient's knee. Other suitable configurations are also possible with more or less than three forces being exerted on the patient's leg.

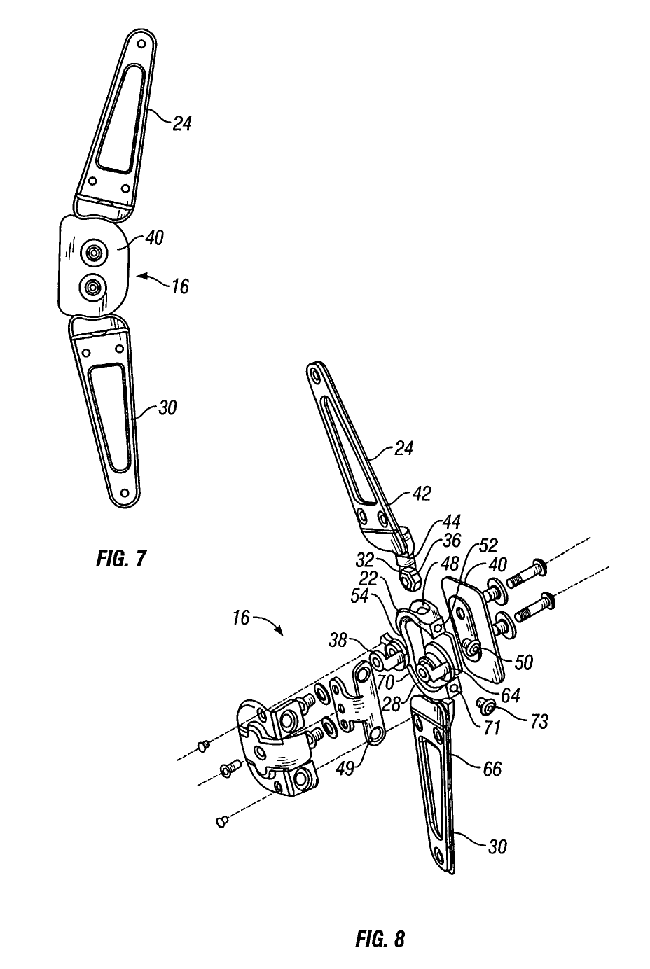

As shown in FIGS. 3-8, the hinge assembly 16 can include an upper ramp 22, an upper arm 24, a flexion/extension hinge 26, a lower ramp 28, and a lower arm 30. FIGS. 1, 2 and 8 illustrate the hinge assembly 16 including a cover over the flexion/extension hinge 26, while FIGS. 3-7 illustrate the hinge assembly 16 without the cover. In some embodiments, the cover is positioned on a lateral side of the hinge assembly 16 with respect to the patient's knee. In one embodiment, the cover is constructed of nylon. In other embodiments, other materials can be used for the construction of the cover, either alone or in combination, such as DELRIN® (E. I. du Pont de Nemours and Company) or its generic equivalent Acetal, Ertalyte® (Quadrant DSM Polymer Corporation), Noryl® (General Electric Company), UHMW Polyethylene or one of its commercial versions Lenite® (Westlake Plastics Company) or Tivar 1000® (Poly Hi Solidue, Inc.), hardened polymers, steel and other metals, metal alloys, plastics, aluminum, fiberglass, aramide fibers, graphite fibers, epoxy resins, polyester resins, polyurethane resins, acrylic resins, carbon-fiber composite, etc.

The arms 24 and 30 are preferably constructed of aluminum or steel; however, the arms 24 and 30 can be constructed of any other suitable metals or non-metal materials. As shown in FIGS. 1-8, the arms 24 and 30 can include triangular-shaped support members 42 and 66, respectively. However, the support members 42 and 66 can have any suitable shape, such as rectangular, oval, trapezoidal, cylindrical, tubular, etc. The support members 42 and 66 can be solid or hollow members, or the support members 42 and 66 can include cut-out portions, holes, or apertures. For example, as shown in FIGS. 1-4, 7 and 8, the support members 42 and 66 include holes for fasteners and a triangular-shaped aperture (which can serve to reduce the weight of the arms 24 and 30, while still providing sufficient strength and rigidity).

The arms 24 and 30 can be coupled to the shells 12 and 14 by sets of screws 46 and 76, respectively. For example, two sets of screws 46 and 76 including three screws each can be used to coupled the arms to the shells. However, the arms 24 and 30 can be coupled to the shells 12 and 14 by any number of releasable or non-releasable fasteners, such as bolts, buckles, clips, mating pins and apertures, nails, rivets, threaded connections, snap-fit connections, press-fit connections, etc. Similarly, adhesives or resins (e.g., epoxy or silicone), cohesive bonding material, welds, and brazing can be used to couple the arms 24 and 30 to the shells 12 and 14. Moreover, various embodiments could employ none, one, or some of these fasteners and methods of attachment in different manners of connection.

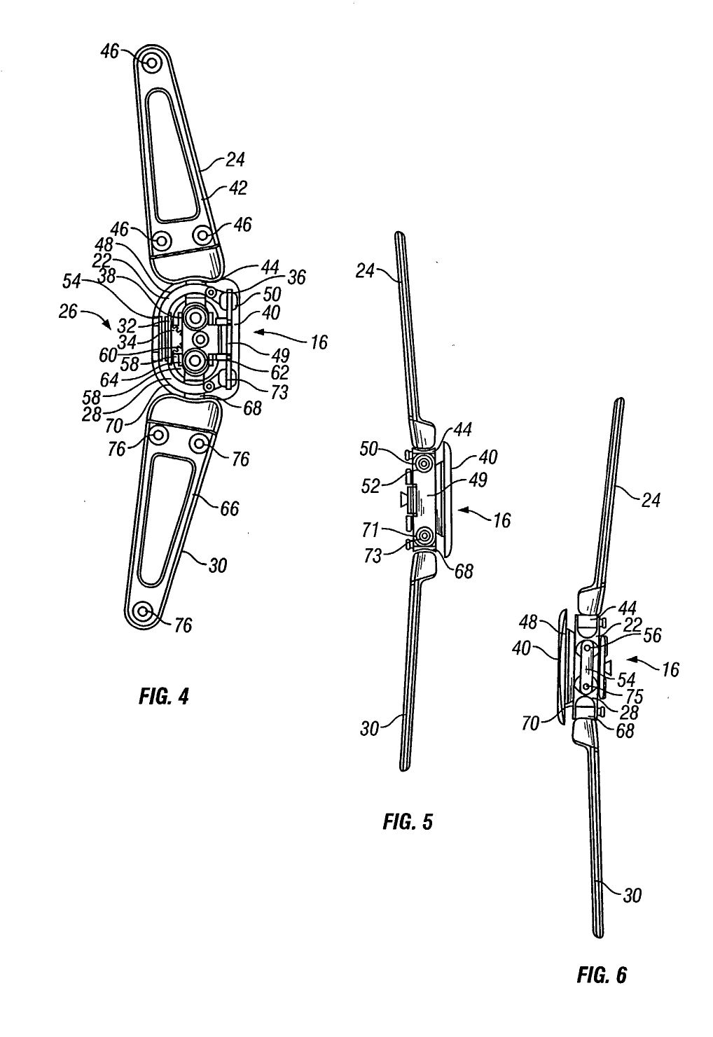

As best shown in FIG. 4, the upper arm 24 can include an end 32 to form an upper portion of the flexion/extension hinge 26. The end 32 can be in the shape of a gear having a plurality of teeth 34. However, in some embodiments the end 32 does not include teeth, but rather can mate with a second component in any other suitable manner. As best shown in FIG. 8, the end 32 of the upper arm 24 can also include a bearing 36, such as a roller bearing or a needle roller bearing. At least a portion of the bearing 32 can be positioned substantially within a bracket or bushing 38 which can be constructed of a metal (such as aluminum, steel, or bronze) or a non-metal material. The bracket 38 can be rotatably coupled to a base plate 40, which can also be constructed of a metal (such as aluminum or steel) or a non-metal (such as nylon) or any other suitable material. In one embodiment, the bearing 36 can be angled medially and laterally with respect to the bracket 38 within a range up to approximately twenty degrees, but preferably ten degrees. In some embodiments, the bracket 38 is positioned on a lateral side and the base plate 40 is positioned on a medial side of the hinge assembly 16 with respect to the patient's knee. The end 32 can be coupled to the support member 42 of the upper arm 24 by an extension 44. The extension 44 can have any suitable cross-sectional shape, such as round, square, triangular, hexagonal, oval, etc.

In some embodiments, the extension 44 of the arm 24 engages the upper ramp 22. The term “ramp” as used herein and in the appended claims generally refers to a structure that includes one or more surfaces shaped and configured to engage a portion of an arm (such as the extension 44) of the knee brace in order to move the arm along a path. In one embodiment, the upper ramp 22 is a ramp cage or cage member including a slot 48. The terms “ramp cage” and “cage member” as used herein and in the appended claims generally refer to a structure including at least two surfaces shaped and configured to engage a portion of an arm of the knee brace and between which the arm moves along a path.

As shown in FIGS. 1-8, the extension 44 can be positioned within the slot 48 defined by the upper ramp 22. In some embodiments, the slot 48 can have an elongated oval shape. The extension 44 can move along an interior surface of the slot 48 as the flexion/extension hinge 26 follows the flexion and extension of the patient's knee.

The slot 48 can be defined so that the unloading force applied to the patient's knee during extension is greater than the unloading force applied during flexion. For example, the distance between the extension 44 and the base plate 40 can be increased or decreased during extension according the contour of the slot 48 in order to apply the appropriate unloading forces to the patient's knee. FIGS. 21A-21D illustrate four examples of positions of the knee brace 10 with respect to the patient's knee and an arthritic compartment. In general, the upper and lower arms 24 and 30 are used as moment arms with respect to the patient's knee. In FIG. 21A, the distance between the extension 44 and the medial base plate 40 is decreased during extension in order to unload an arthritic knee compartment on the lateral side of the patient's knee. In FIG. 21B, the distance between the extension 44 and the medial base plate 40 is increased during extension in order to unload an arthritic knee compartment on the medial side of the patient's knee. In FIG. 21C, the distance between the extension 44 and the medial base plate 40 is decreased during extension in order to unload an arthritic knee compartment on the medial side of the patient's knee. In FIG. 21D, the distance between the extension 44 and the medial base plate 40 is increased during extension in order to unload an arthritic knee compartment on the lateral side of the patient's knee.

In some embodiments, the change in the force applied to the patient's knee is gradual due to a smooth contour of an interior surface of the slot 48 in the upper ramp 22. However, the change in the force applied to the patient's knee can also occur in one or more steps or any other suitable path or pattern. For example, the forces applied to the patient's leg can be increased just before the patient's knee is fully extended, and then decreased as soon as the patient's knee begins to flex. In this manner, the knee brace 10 can apply a maximum unloading force to the patient's knee during extension, but reduce the unloading force as soon as flexion begins.

In some embodiments, an anterior side of the upper ramp 22 is coupled to an anterior base plate 49 that is connected to the medial base plate 40. The upper arm 22 can be pivotably coupled to the medial base plate 40 by a screw 50 positioned within an aperture 52 in the anterior base plate 49. The screw 50 can include a hexagonal screw head that is rotatable with an Allen wrench. The aperture 52 is preferably elongated so that the upper ramp 22 can be moved medially and laterally in order to adjust the unloading force. A posterior side of the upper ramp 22 can also be pivotably coupled to a posterior base plate 54 that is connected to the medial base plate 40. The upper ramp 22 is pivotably coupled to the posterior base plate 54 by a second screw 56. Due to the pivotable upper ramp 22, the medial/lateral orientation of the upper ramp 22 can be changed with respect to the flexion/extension hinge 26.

In other embodiments, the upper ramp 22 is rigidly coupled to the anterior base plate 49 by a one way or non-reversible screw 50 positioned within the aperture 52 in the anterior base plate 49. In these embodiments, the aperture 52 is preferably a hole through which the upper ramp 22 is secured in one medial/lateral position. In addition, the non-reversible screw 50 can be coated with a thread-locking compound, such as LOCTITE®, in order to further secure the one-way screw 50 within the aperture 52. In some embodiments, the non-reversible screw 50 includes a hexagonal screw head filled with an epoxy resin (or any other suitable material) so that the screw head is not accessible with an Allen wrench. In addition, the cover for the hinge assembly 16 can be constructed to extend over the non-reversible screw 50. The posterior side of the upper ramp 22 can also be rigidly coupled to a posterior base plate 54 that is connected to the medial base plate 40. The upper ramp 22 can be rigidly coupled to the posterior base plate 54 by a second screw 56. Due to the first non-reversible screw 50 and the thread locking compound, the medial/lateral orientation of some embodiments of the upper ramp 22 cannot be adjusted with respect to the flexion/extension hinge 26.

Similar to the upper arm 24, the lower arm 30 can include an end 58 to form a lower portion of the flexion/extension hinge 26. The end 58 can be in the shape of a gear having a plurality of teeth 60. However, in some embodiments the end 60 does not include teeth but rather mates with the upper arm 24 in another suitable manner. As best shown in FIG. 8, the end 58 of the lower arm 30 can also include a bearing 62, such as a roller bearing or a needle roller bearing. At least a portion of the bearing 62 can be positioned substantially within a bracket or bushing 64. The bracket 64 can be rotatably coupled to the base plate 40. In one embodiment, the bearing 62 can be angled medially and laterally with respect to the bracket 64 within a range up to approximately twenty degrees, but preferably ten degrees. In some embodiments, the bracket 64 is positioned on a lateral side and the base plate 40 is positioned on a medial side of the hinge assembly 16 with respect to the patient's knee. The end 58 can be coupled to the support member 66 of the lower arm 30 by an extension 68. The extension 68 can have any suitable cross-sectional shape, such as round, square, triangular, hexagonal, oval, etc.

In some embodiments, the extension 68 of the arm 30 engages the lower ramp 28. In one embodiment, the lower ramp 28 is a ramp cage including a slot 70. As shown in FIGS. 1-8, the extension 68 can be positioned within the slot 70 defined by the lower ramp 28. The extension 68 can move along an interior surface of the slot 70 as the flexion/extension hinge 26 follows the flexion and extension of the patient's knee. As described above with respect to the upper ramp 22 and the slot 48, the slot 70 can be elongated, oval and/or defined so that the force applied to the patient's knee during extension is greater or less than the force applied during flexion. In some embodiments, the change in the force applied to the patient's knee is gradual due to a smooth contour of an interior surface of the slot 70 in the lower ramp 28.

In some embodiments, an anterior side of the lower ramp 28 is coupled to the anterior base plate 49 that is connected to the medial base plate 40. The lower arm 30 can be pivotably coupled to the medial base plate 40 by a screw 73 positioned within an aperture 71 in the anterior base plate 49. The screw 50 can include a screw head that is rotatable with an Allen wrench. The aperture 71 is preferably elongated so that the lower ramp 28 can be moved medially and laterally in order to adjust the unloading force. A posterior side of the lower ramp 28 can also be pivotably coupled to the posterior base plate 54 that is connected to the medial base plate 40. The lower ramp 28 can be pivotably coupled to the posterior base plate 54 by a second screw 75. As a result, the medial/lateral orientation of the lower ramp 28 can preferably be adjusted with respect to the flexion/extension hinge 26. In other embodiments, as described above with respect to the upper ramp 22, the lower ramp 28 is rigidly coupled to the anterior base plate 49 by a one way or non-reversible screw 73 positioned within the aperture 71 in the anterior base plate 49.

Thus, in one preferred embodiment, the medial/lateral orientation of both the upper ramp 22 and the lower ramp 28 can be adjusted with respect to the flexion/extension hinge 26. The medial/lateral orientation of the upper and lower ramps 22 and 28 can be adjusted by loosening the screws 50 and 73 (in some embodiments, with an Allen wrench), moving the anterior sides of the upper and lower ramp cages 22 and 28 with respect to the apertures 52 and 71 so that the posterior sides of the upper and lower ramps 22 and 28 rotate with respect to the posterior base plate 54, and then re-tightening the screws 50 and 73. However, in other embodiments, only one or neither of the upper ramp 22 and the lower ramp 28 are adjustable medially and laterally.

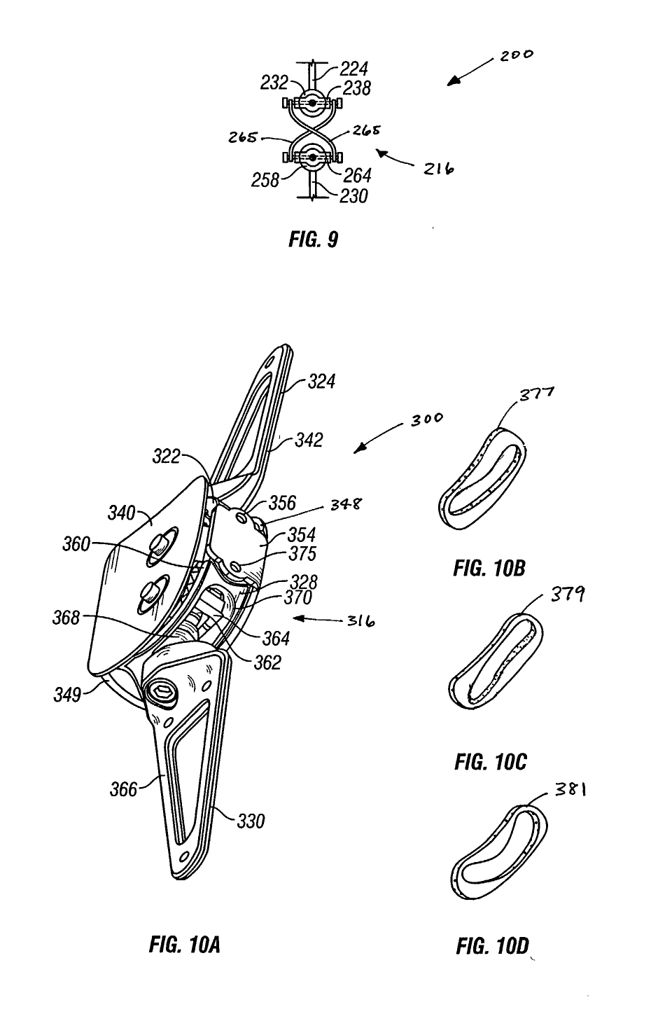

FIG. 9 illustrates a portion of a knee brace 200 which is an alternative embodiment of the knee brace 10 shown in FIGS. 1-8. Elements and features of the knee brace 200 illustrated in FIG. 9 having a form, structure, or function similar to that found in the knee brace 10 of FIGS. 1-8 are given corresponding reference numbers in the 200 series. The knee brace 200 includes an upper arm 224 coupled to a lower arm 230 by a hinge assembly 216. The hinge assembly 216 can include an upper bearing 232, an upper bracket 238, a lower arm 230, a lower bearing 258, and a lower bracket 264. Rather than or in addition to having geared ends, the upper bracket 238 can be coupled to the lower bracket 264 with one or more tension straps 265. As shown in FIG. 9, the tension straps 265 can be arranged to cross one another in a figure-eight configuration. In other embodiments, one tension strap 265 can be positioned on a posterior or anterior side of the upper and lower brackets 238 and 264 in order to provide either preferential extension or flexion. In general, the addition of one or more tension straps 265 in any suitable configuration can provide additional stability to the hinge assembly 216. The tension straps can be constructed of any suitable flexible material, such as polyurethane. In general, the material for the tension straps 265 preferably has little or no elasticity, high flexibility, and high tensile strength.

FIGS. 10A-10D illustrate a knee brace 300 which is an alternative embodiment of the knee braces 10 and 200. Elements and features of the knee brace 300 illustrated in FIGS. 10A-10D having a form, structure, or function similar to that found in the knee braces of FIGS. 1-9 are given corresponding reference numbers in the 300 series. As shown in FIG. 10A, the knee brace 300 includes an upper arm 324 (including a triangular support member 342) coupled to a lower arm 330 (including a triangular support member 366) by a hinge assembly 316. The hinge assembly 316 can include an upper ramp 322 with a slot 348 and a lower ramp 328 with a slot 370. The hinge assembly 316 also includes a medial base plate 340, an anterior base plate 349, and posterior base plate 354 (through which screws 356 and 375 can be positioned). The lower arm 330 includes a plurality of teeth 360 and a bearing 362 positioned in a bracket 364. The lower arm 330 is coupled to an extension 368 that moves within the slot 370. As shown in FIGS. 10B-10D, the knee brace 300 can include inserts 377, 379 and 381 that can be selectively positioned within the slot 370. In some embodiments, the inserts can be removably positioned within the slot 370. The inserts each include a different aperture to define two contoured surfaces between which the extension 368 moves in order to provide different unloading forces to the patient's knee. For example, the insert 377 moves the arm 330 toward the medial base plate 340 during extension and the insert 379 moves the arm 330 away from the medial base plate 340 during extension. The insert 381 moves the arm 330 toward the medial base plate 340 during full flexion, then away during partial flexion, and then back toward the medial base plate 340 during full extension. However, the inserts can have any suitable configuration in order to provide the appropriate forces to the patient's leg. Moreover, rather than including inserts, the slots 348 and 370 of the ramps 322 and 328 can include contoured surfaces shaped according to the various configurations of the inserts.

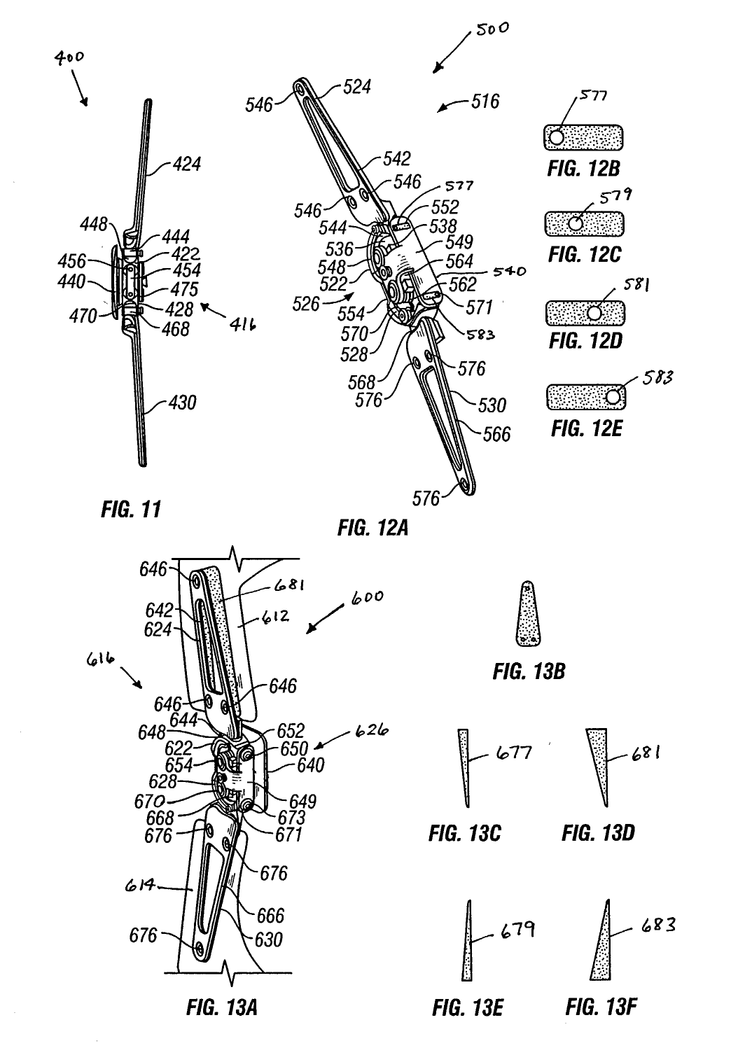

FIG. 11 illustrates a knee brace 400 which is an alternative embodiment of the knee braces 10, 200 and 300. Elements and features of the knee brace 400 illustrated in FIG. 11 having a form, structure, or function similar to that found in the knee braces of FIGS. 1-10D are given corresponding reference numbers in the 400 series. As shown in FIG. 11, the knee brace 400 includes an upper arm 424 coupled to a lower arm 430 by a hinge assembly 416. The hinge assembly 416 includes a medial base plate 440 and posterior base plate 454 (through which screws 456 and 475 can be positioned). The hinge assembly 416 can include an upper ramp 422 with a single contoured surface 448 and a lower ramp 428 with a single contoured surface 470 (rather than the slots of the ramp cages described above with respect to the knee braces 10, 200 and 300). The upper arm 424 is coupled to an extension 444 that moves along the contoured surface 448 and the lower arm 430 is coupled to an extension 468 that moves along the contoured surface 470. The contoured surfaces 448 and 470 are preferably elongated. In this embodiment, the hinge assembly 416 must be positioned with respect to the patient's knee so that the arms 424 and 430 are forced to engage the upper and lower ramps 422 and 428.

FIGS. 12A-12E illustrate a knee brace 500 which is an alternative embodiment of the knee braces 10, 200, 300 and 400. Elements and features of the knee brace 500 illustrated in FIGS. 12A-12E having a form, structure, or function similar to that found in the knee braces of FIGS. 1-11 are given corresponding reference numbers in the 500 series. As shown in FIG. 12A, the knee brace 500 includes an upper arm 524 (including a triangular support member 542 and fasteners 546) coupled to a lower arm 530 (including a triangular support member 566 and fasteners 576) by a hinge assembly 516 including a flexion/extension hinge 526. The hinge assembly 516 can include an upper ramp 522 with a slot 548 and a lower ramp 528 with a slot 570. The upper arm 524 is coupled to an extension 544 that moves within the slot 548, and the lower arm 530 is coupled to an extension 568 that moves within the slot 570. The upper arm 524 includes a bearing 536 positioned in a bracket 538, and the lower arm 530 includes a bearing 562 positioned in a bracket 564. The hinge assembly 516 also includes an anterior base plate 549 and a posterior base plate 554. The anterior base plate 549 includes two elongated apertures 552 and 571. The anterior base plate 549 can be increased in size in order to accommodate larger apertures 552 and 571. As shown in FIGS. 12B-10E, the knee brace 500 can include inserts 577, 579, 581 and 583 that can be selectively positioned within each of the apertures 552 and 571. The inserts can be constructed of a metal, a reinforced or non-reinforced plastic, or any other suitable material. Preferably, the inserts are interchangeable (i.e., the same insert can be positioned in the apertures or a different insert can be positioned in each aperture). In some embodiments, the inserts can be removably positioned within the apertures 552 and 571, so that different inserts can be used if necessary. Each insert can include a hole at a different location along the length of the insert to define the medial/lateral position of the corresponding arm 524 or 530. For example, as shown in FIG. 12A, the insert 577 results in the arm 524 being inclined away from the medial base plate 540, and the insert 583 results in the arm 530 being inclined toward the medial base plate 540. By way of example only, the inserts 579 and 581 result in inclinations between the two extremes that result from the inserts 577 and 583. However, the inserts can have any suitable configuration in order to provide the appropriate forces to the patient's leg.

FIGS. 13A-13E illustrate a knee brace 600 which is an alternative embodiment of the knee braces 10, 200, 300, 400 and 500. Elements and features of the knee brace 600 illustrated in FIGS. 13A-13E having a form, structure, or function similar to that found in the knee braces of FIGS. 1-12E are given corresponding reference numbers in the 600 series. As shown in FIG. 13A, the knee brace 600 includes an upper arm 624 (including a triangular support member 642 and fasteners 646) coupled to a lower arm 630 (including a triangular support member 666 and fasteners 676) by a hinge assembly 616 including a flexion/extension hinge 626. The upper arm 624 is coupled to an upper shell 612 and the lower arm 630 is coupled to a lower shell 614. The hinge assembly 616 can include an upper ramp 622 with an elongated oval slot 648 and a lower ramp 628 with an elongated oval slot 670. The upper arm 624 is coupled to an extension 644 that moves within the elongated oval slot 648, and the lower arm 630 is coupled to an extension 668 that moves within the elongated oval slot 670. The hinge assembly 616 also includes a medial base plate 640 and a posterior base plate 654. The anterior base plate 649 includes two screws 650 and 673 positioned within two apertures 652 and 671. As shown in FIGS. 13B-13F, the knee brace 600 can include wedges 677, 679, 681 and 683 that can be selectively positioned between the upper and lower shells 612 and 614 and the upper and lower arms 624 and 630. The wedges can be constructed of any type of metal or plastic or any other suitable material having sufficient compressive strength. Preferably, the wedges are interchangeable (i.e., the same wedge can be positioned between the upper and lower arms and shells or a different wedge can be positioned between the upper and lower arms and shells). In some embodiments, the wedges can be removably positioned between the upper and lower arms and shells, so that different wedges can be used if necessary. As shown in the front view of a wedge in FIG. 13B, the wedges can be shaped to correspond to the shape of the arms 624 and 630 and include holes through which the fasteners 646 and 676 can be positioned. Although not shown in FIG. 13B, the wedges can include a triangular-shaped, cut-out portion corresponding to the cut-out portion of the arms 624 and 630. As shown in the side views of the wedges in FIGS. 13C-13F, the wedges can each include a different sloped surface to define the medial/lateral position of the arm 624 or 630 under which the wedge is positioned. For example, as shown in FIG. 13A, the wedge 681 results in the arm 624 being inclined about ten degrees laterally with respect to the medial base plate 640. By way of example only and with respect to the base plate 640 being positioned medially with respect to the patient's knee, the wedge 677 results in an approximately five degree lateral inclination, the wedge 679 results in an approximately five degree medial inclination, the wedge 681 results in an approximately ten degree lateral inclination, and the wedge 683 results in an approximately ten degree medial inclination. However, the wedges can include any suitable sloped surface in order to provide the appropriate forces to the patient's leg.

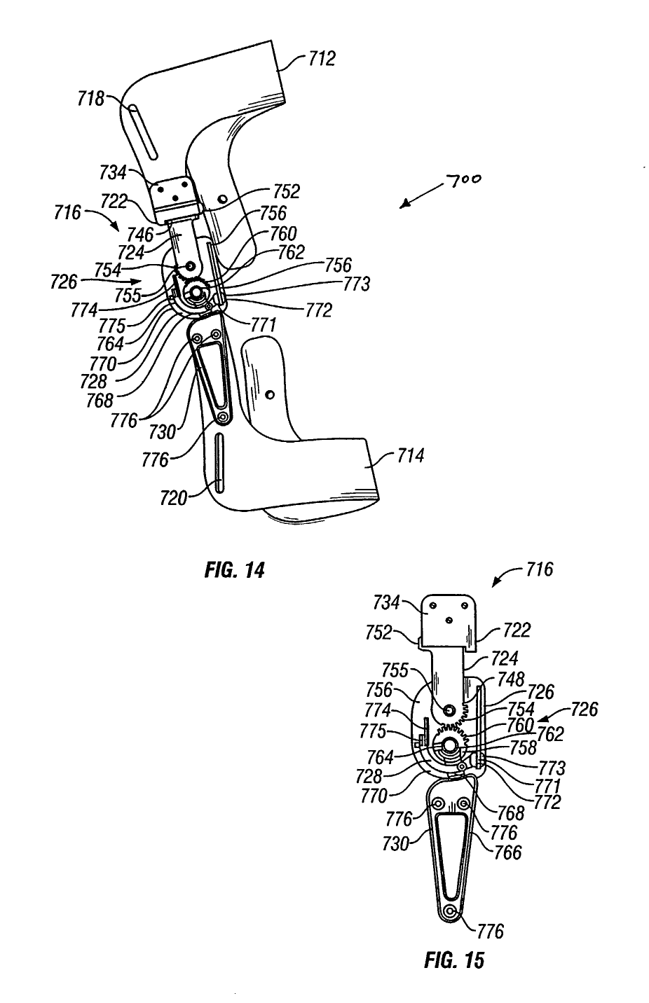

FIGS. 14-19 illustrate a knee brace 700 which is an alternative embodiment of the knee braces 10, 200, 300, 400, 500 and 600. Elements and features of the knee brace 700 illustrated in FIGS. 14-19 having a form, structure, or function similar to that found in the knee braces of FIGS. 1-13F are given corresponding reference numbers in the 700 series. As shown in FIG. 14, the knee brace 700 includes an upper shell 712 coupled to a lower shell 714 by a hinge assembly 716. The upper shell 712 and the lower shell 714 are secured to the patient's right or left leg with two or more straps (not shown) positionable through an upper slot 718 and a lower slot 720. The side of the knee brace 700 that includes the hinge assembly 716 can be positioned on either side of the patient's knee with respect to the knee compartment having osteoarthritis.

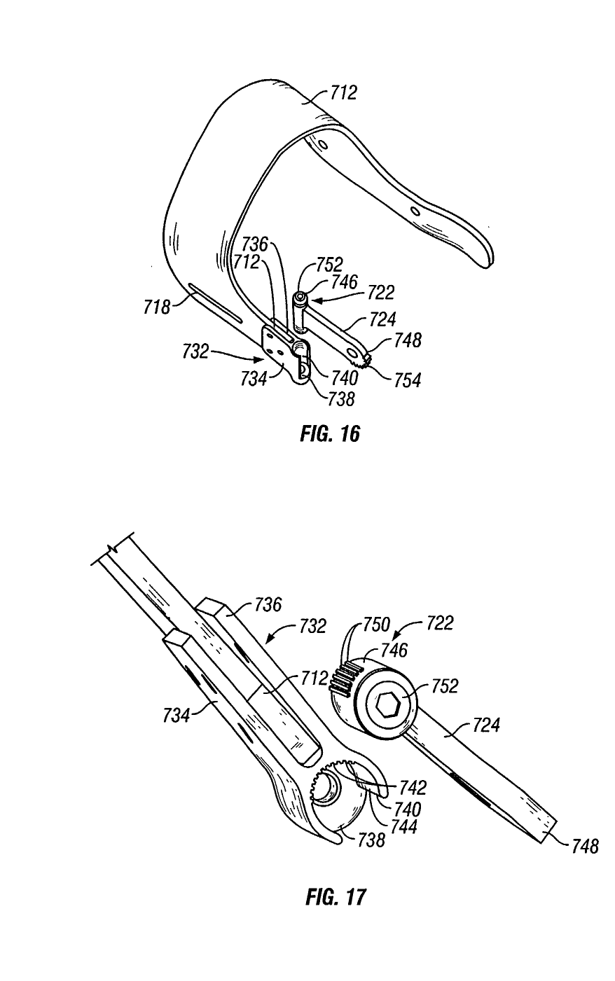

As shown in FIGS. 14 and 15, the hinge assembly 716 includes a cylinder joint 722, an upper arm 724, a flexion/extension hinge 726, a ramp 728, and a lower arm 730. As shown in FIGS. 16 and 17, the cylinder joint 722 can include a bracket 732 connected to the upper shell 712. The bracket 732 can include a lateral portion 734 connected to the lateral side of the upper shell 712 and a medial portion 736 connected to the medial side of the upper shell 712. The bracket 732 can include a cylindrical receiving aperture 738, which can include a cut-out portion 740. As shown in FIG. 17, a plurality of slots 742 can be defined on an interior surface 744 of the bracket 732 within the cylindrical receiving aperture 738. The upper arm 724 can include a first cylindrical end 746 and a second end 748 (which can be a gear including teeth 754). The first cylindrical end 746 can be sized to be received within the cylindrical receiving aperture 738 of the bracket 732. The first cylindrical end 746 of the upper arm 724 can include a plurality of ridges 750 to engage the plurality of slots 742 on the interior surface 744 of the bracket 732. In other embodiments, the interior surface 744 can include a locking taper configuration (e.g., a Morse taper) and the first cylindrical end 746 can include a corresponding configuration to engage the locking taper of the interior surface 744. The first cylindrical end 746 of the upper arm 724 can be secured within the cylindrical receiving aperture 738 preferably with a removable retaining bolt 752. However, in the embodiments that include a locking taper, the retaining bolt 754 can be omitted. As shown in FIGS. 14 and 16, the second end 748 of the upper arm 724 can form an upper portion of the flexion/extension hinge 726. A bolt 755 can be positioned through the center of the second end 748 in order to rotatably couple the second end 748 to a medial base plate 756.

As shown in FIGS. 14 and 15, the lower arm 730 can include a first end 758 preferably having a plurality of teeth 760 and forming a lower portion of the flexion/extension hinge 726. The plurality of teeth 760 of the lower arm 730 can engage the plurality of teeth 754 of the upper arm 724. The first end 758 of the lower arm 730 can include a bearing 762 positioned within a bracket 764 coupled to the base plate 756. The first end 758 can also be coupled to a triangular support member 766 of the lower arm 30 by an extension 768. The triangular support member 766 of the lower arm 730 can be coupled to the lower shell 714 by fasteners 776.

The extension 768 is positioned within a slot 770 defined by the ramp 728. The extension 768 moves along an interior surface of the slot 770 as the flexion/extension hinge 726 follows the flexion and extension of the patient's knee. The ramp cage 28 can be movably coupled to a base plate 772 that is connected to the base plate 756. The ramp 728 can be movably coupled to the anterior base plate 772 by a first bolt 773 positioned within an aperture 771 in the base plate 772. The ramp 728 can also be rotatably coupled to a base plate 774 that is connected to the base plate 756. The ramp 728 can be rotatably coupled to the base plate 774 by a second bolt 775.

As shown in FIGS. 14, 16 and 17, the medial/lateral orientation of the ramp cage 728 can be adjusted with respect to the flexion/extension hinge 726. The medial/lateral orientation of the ramp cage 728 can be adjusted by loosening the first bolt 773, moving the ramp 728 with respect to the aperture 771 in the base plate 772 so that the ramp 728 rotates with respect to the base plate 774, and then re-tightening the first bolt 773. Regardless of the medial/lateral orientation of the ramp 728, the slot 770 is defined so that more or less pressure is applied to the patient's knee during extension than during flexion. The change between applying more or less pressure to the patient's knee is preferably a gradual change due to the smooth contour of the interior surface of the slot 770 in the ramp 728.

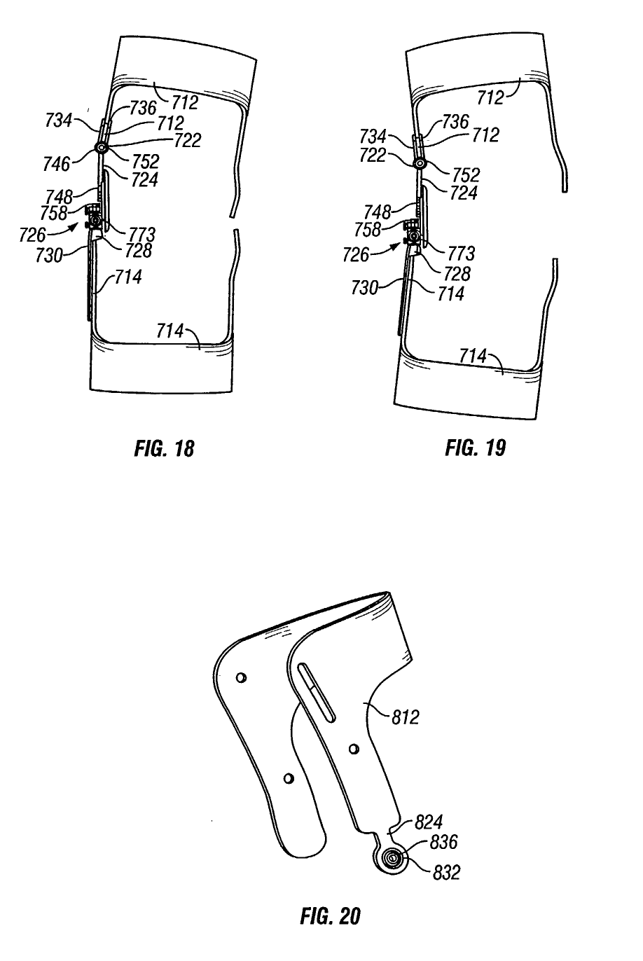

As also shown in FIGS. 14, 16 and 17, the medial/lateral orientation of the upper arm 724 can be adjusted with respect to the upper shell 712, rather than with respect to the flexion/extension hinge 726. In order to change the medial/lateral orientation of the upper arm 724 with respect to the upper shell 712, the upper arm 724 must be disassembled from the upper shell 712. Specifically, the retaining bolt 752 is removed from the first cylindrical end 746 of the upper arm 724. As shown in FIG. 16, the first cylindrical end 746 of the upper arm 724 is then removed from the cylindrical receiving aperture 738. As shown in FIG. 17, the first cylindrical end 746 of the upper arm 724 can be inserted into a different position within the cylindrical receiving aperture 738 so that the plurality of ridges 750 engage the plurality of slots 742 in a different manner. The degree of adjustability for the medial/lateral orientation of the upper arm 724 with respect to the upper shell 712 is generally defined by the width of the cut-out portion 740 of the bracket 732. As shown in FIG. 18, if the knee brace 700 is positioned on the patient's right leg, the upper shell 712 has been angled medially with respect to the upper arm 724. As shown in FIG. 19, the upper shell 712 has been angled laterally with respect to the upper arm 724.

It should be understood that the configurations for the attachment of the upper arm 724 to the upper shell 712 and the attachment of the lower arm 730 to the lower shell 714 can be reversed. In other words, the cylindrical joint 722 can be used in conjunction with the lower arm 730 and the ramp 728 can be used in conjunction with the upper arm 724. Also, two cylindrical joints can be used to attach the flexion/extension hinge 726 to the upper shell 712 and the lower shell 714. In those embodiments, the inclination of both the upper and lower arms can be adjusted with respect to the upper and lower shells, rather than with respect to the flexion/extension hinge 726.

FIG. 20 illustrates an alternative embodiment of an upper shell 812. In this embodiment, an upper arm 824 is integral with the upper shell 812. In addition, the upper arm 824 includes an end 832 having a bearing 836. The upper arm 824 can be constructed of the same material as the upper shell 812, but the upper arm 824 is preferably reinforced with a metal or another material having sufficient strength and rigidity. A lower shell can also include an integral lower arm, or only one of the lower shell and the upper shell can include an integral arm.

It should also be understood that the knee braces 10, 200, 300, 400, 500, 600 and 700 described above can be used to treat other knee conditions, such as anterior cruciate ligament (ACL) injuries, and are not limited to use as a treatment for osteoarthritis. In addition, the hinge assemblies 16, 216, 316, 416, 516, 616 and 716 described above can be used in other orthotic braces, such as hip, elbow or shoulder orthoses. For example, a hip orthosis can include only a lower ramp and a lower arm that moves along the lower ramp. Also, an elbow orthosis can include a proximal ramp (i.e., a ramp closer to the patient's shoulder), a proximal arm, a distal ramp (i.e., a ramp closer to the patient's hand), and a distal arm.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

We claim:

1. A knee brace for coupling to a patient's knee, the knee brace comprising:
a flexion/extension joint;
a first arm coupled to the flexion/extension joint; and
a first ramp positioned to engage the first arm, the first ramp shaped to move the first arm towards the patient's leg as the patient's knee extends and away from the patient's leg as the patient's knee flexes.

2. The knee brace of claim 1, wherein the first ramp includes a contoured surface shaped to move the first arm gradually.

3. The knee brace of claim 2, wherein the contoured surface is shaped to move the first arm up to approximately twenty degrees with respect to the flexion/extension joint.

4. The knee brace of claim 2, wherein the first ramp includes a cage member with a first aperture through which the first arm is positioned.

5. The knee brace of claim 4, wherein the first aperture of the cage member defines the contoured surface.

6. The knee brace of claim 4, further comprising a cage member insert with a second aperture, the insert including a portion coupled within the first aperture of the cage member, and wherein the second aperture defines the contoured surface.

7. The knee brace of claim 1, wherein the first ramp includes a single plate along which the first arm moves.

8. The knee brace of claim 1, wherein:
the flexion/extension joint includes a base plate;
the first ramp is pivotably coupled to the base plate; and
the first ramp is pivotable medially and laterally with respect to the base plate and the patient's knee.

9. The knee brace of claim 8, wherein the base plate is positioned anteriorly with respect to the flexion/extension joint.

10. The knee brace of claim 8, wherein the first arm includes a bearing positioned within a bracket, and wherein the first arm pivots medially and laterally about the bearing when the first ramp pivots medially and laterally.

11. The knee brace of claim 8, further comprising a base plate insert having a hole through which a fastener is positionable, wherein the hole is located in one of a plurality of positions along a length of the base plate insert, and wherein the base plate insert includes a portion coupled within an aperture in the base plate and the fastener is positionable in the hole in order to secure the first ramp medially and laterally.

12. The knee brace of claim 1, further comprising:
a second arm coupled to the flexion/extension joint; and
a second ramp positioned to engage the second arm, the second ramp shaped to move the second arm toward the patient's leg as the patient's knee extends and away from the patient's leg as the patient's knee flexes.

13. The knee brace of claim 12, wherein the first arm is coupled to a first gear and the second arm is coupled to a second gear that engages the first gear.

14. The knee brace of claim 13, wherein the first arm includes a first bearing at least partially positioned within a first bracket coupled to the first gear and the second arm includes a second bearing at least partially positioned within a second bracket coupled to the second gear.

15. The knee brace of claim 14, wherein the first arm pivots medially and laterally about the first bearing when the first ramp pivots medially and laterally and the second arm pivots medially and laterally about the second bearing when the second ramp pivots medially and laterally.

16. The knee brace of claim 12, wherein the first arm pivots posteriorly and anteriorly about a first pivot point and the second arm pivots posteriorly and anteriorly about a different second pivot point when the patient's knee flexes and extends.

17. The knee brace of claim 12, wherein:
the flexion/extension joint includes a base plate;
the first ramp and the second ramp are each pivotably coupled to the base plate; and
the first ramp and the second ramp are each independently pivotable medially and laterally with respect to the base plate and the patient's knee.

18. The knee brace of claim 12, wherein the flexion/extension joint includes at least one tension strap.

19. The knee brace of claim 1, further comprising a first shell to which the first arm is secured and a wedge including a sloped surface positionable between the first arm and the first shell.

20. The knee brace of claim 1, further comprising a first shell, and wherein the first arm is secured to the first shell with a cylindrical joint.

21. The knee brace of claim 20, wherein the cylindrical joint allows the first arm to be positioned medially and laterally with respect to the first shell.

22. The knee brace of claim 20, wherein the cylindrical joint includes a bracket coupled to the first shell and having a cylindrical aperture, and wherein the first arm includes a cylindrical end at least partially positionable within the cylindrical aperture of the bracket.

23. The knee brace of claim 1, and further comprising a first shell and wherein the first arm is integral with the first shell.

24. A knee brace for coupling to a patient's knee, the knee brace comprising:
a flexion/extension joint;
a first arm coupled to the flexion/extension joint; and
a first ramp positioned to engage the first arm, the first ramp shaped to move the first arm away from the patient's leg as the patient's knee flexes and towards the patient's leg as the patient's knee extends.

25. The knee brace of claim 24, wherein the first ramp includes a contoured surface shaped to move the first arm gradually.

26. The knee brace of claim 25, wherein the contoured surface is shaped to move the first arm up to approximately twenty degrees with respect to the flexion/extension joint.

27. The knee brace of claim 25, wherein the first ramp includes a cage member with a first aperture through which the first arm is positioned.

28. The knee brace of claim 27, wherein the first aperture of the cage member defines the contoured surface.

29. The knee brace of claim 27, further comprising a cage member insert with a second aperture, the insert including a portion coupled within the first aperture of the cage member, and wherein the second aperture defines the contoured surface.

30. The knee brace of claim 24, wherein the first ramp includes a single plate along which the first arm moves.

31. The knee brace of claim 24, wherein:
the flexion/extension joint includes a base plate;
the first ramp is pivotably coupled to the base plate; and
the first ramp is pivotable medially and laterally with respect to the base plate and the patient's knee.

32. The knee brace of claim 31, wherein the base plate is positioned anteriorly with respect to the flexion/extension joint.

33. The knee brace of claim 31, wherein the first arm includes a bearing positioned within a bracket, and wherein the first arm pivots medially and laterally about the bearing when the first ramp pivots medially and laterally.

34. The knee brace of claim 31, further comprising a base plate insert having a hole through which a fastener is positionable, wherein the hole is located in one of a plurality of positions along a length of the base plate insert, and wherein the base plate insert includes a portion coupled within an aperture in the base plate and the fastener is positionable in the hole in order to secure the first ramp medially and laterally.

35. The knee brace of claim 24, further comprising:
a second arm coupled to the flexion/extension joint; and
a second ramp positioned to engage the second arm, the second ramp shaped to move the second arm toward the patient's leg as the patient's knee extends and away from the patient's leg as the patient's knee flexes.

36. The knee brace of claim 35, wherein the first arm is coupled to a first gear and the second arm is coupled to a second gear that engages the first gear.

37. The knee brace of claim 36, wherein the first arm includes a first bearing at least partially positioned within a first bracket coupled to the first gear and the second arm includes a second bearing at least partially positioned within a second bracket coupled to the second gear.

38. The knee brace of claim 37, wherein the first arm pivots medially and laterally about the first bearing when the first ramp pivots medially and laterally and the second arm pivots medially and laterally about the second bearing when the second ramp pivots medially and laterally.

39. The knee brace of claim 35, wherein the first arm pivots posteriorly and anteriorly about a first pivot point and the second arm pivots posteriorly and anteriorly about a different second pivot point when the patient's knee flexes and extends.

40. The knee brace of claim 35, wherein:
the flexion/extension joint includes a base plate;
the first ramp and the second ramp are each pivotably coupled to the base plate; and
the first ramp and the second ramp are each independently pivotable medially and laterally with respect to the base plate and the patient's knee.

41. The knee brace of claim 35, wherein the flexion/extension joint includes at least one tension strap.

42. The knee brace of claim 24, further comprising a first shell to which the first arm is secured and a wedge including a sloped surface positionable between the first arm and the first shell.

43. The knee brace of claim 24, further comprising a first shell, and wherein the first arm is secured to the first shell with a cylindrical joint.

44. The knee brace of claim 43, wherein the cylindrical joint allows the first arm to be positioned medially and laterally with respect to the first shell.

45. The knee brace of claim 43, wherein the cylindrical joint includes a bracket coupled to the first shell and having a cylindrical aperture, and wherein the first arm includes a cylindrical end at least partially positionable within the cylindrical aperture of the bracket.

46. The knee brace of claim 24, and further comprising a first shell and wherein the first arm is integral with the first shell.

47. A method of bracing a patient's knee, the method comprising:
securing a brace to the patient's knee;
allowing the patient's knee to flex and extend;
exerting a medial/lateral force on the patient's leg as the patient's knee flexes and extends, the medial/lateral force being exerted on at least one of a first point generally above the patient's knee, a second point generally at the patient's knee, and a third point generally below the patient's knee;
increasing the medial/lateral force as the patient's knee extends; and
decreasing the medial/lateral force as the patient's knee flexes.

48. The method of claim 47, and further comprising gradually increasing the medial/lateral force as the patient's knee extends and gradually decreasing the medial/lateral force as the patient's knee flexes by changing an angle of incidence of the medial/lateral force with respect to the patient's leg by up to approximately twenty degrees.

49. The method of claim 47, wherein the brace includes a flexion/extension joint and an first arm and a second arm coupled to the flexion/extension joint, and further comprising adjusting at least one of the medial/lateral force exerted on the first point by changing a medial/lateral inclination of the first arm with respect to the flexion/extension joint and the medial/lateral force on the third point by changing a medial/lateral inclination of the second arm with respect to the flexion/extension joint.

50. A knee brace for coupling to a patient's knee, the knee brace comprising:
a pair of arms for coupling to the patient's knee; and
a joint between the arms to allow flexion and extension of the patient's knee, a medial/lateral distance between at least one arm of the pair of arms and the patient's leg being greater during flexion than during extension.

51. The knee brace of claim 50, wherein the medial/lateral distance gradually increases as the patient's knee flexes and gradually decreases as the patient's knee extends as an angle of incidence of the at least one arm with respect to the joint changes up to approximately twenty degrees.

52. A knee brace for attachment to a patient's knee, the knee brace comprising:
a pair of arms for coupling to the patient's knee; and
a joint between the arms to allow flexion and extension of the patient's knee, a medial/lateral distance between at least one arm of the pair of arms and the patient's leg being greater during extension than during flexion.

53. The knee brace of claim 52, wherein the medial/lateral distance gradually decreases as the patient's knee flexes and gradually increases as the patient's knee extends as an angle of incidence of the at least one arm with respect to the joint changes up to approximately twenty degrees.

54. A knee brace for coupling to a patient's knee, the knee brace comprising:
a pair of arms for coupling to the patient's knee;
a joint coupled to the pair of arms to allow flexion and extension of the patient's knee, the joint positioned with respect to the patient's knee and configured so that at least one arm of the pair of arms moves laterally as the patient's knee flexes and medially as the patient's knee extends.

55. A knee brace for coupling to a patient's knee, the knee brace comprising:
a pair of arms for coupling to the patient's knee;
a joint coupled to the pair of arms to allow flexion and extension of the patient's knee, the joint positioned with respect to the patient's knee and configured so that at least one arm of the pair of arms moves medially as the patient's knee flexes and laterally as the patient's knee extends.

56. A knee brace for coupling to a patient's knee, the knee brace comprising:
a pair of arms for coupling to the patient's knee;
a first joint coupled to the pair of arms to allow flexion and extension of the patient's knee; and
a second joint in the brace for allowing controlled medial and lateral inclination relative to the first joint of at least one arm of the pair of arms, the medial/lateral inclination of the at least one arm being more lateral during flexion and more medial during extension.

57. A knee brace for coupling to a patient's knee, the knee brace comprising:
a pair of arms for coupling to the patient's knee;
a first joint coupled to the pair of arms to allow flexion and extension of the patient's knee; and
a second joint in the brace for allowing controlled medial and lateral inclination relative to the first joint of at least one arm of the pair of arms, the medial/lateral inclination of the at least one arm being more medial during flexion and more lateral during extension.

58. A knee brace for coupling to a patient's knee, the knee brace comprising:
a pair of arms for coupling to the patient's knee;
means coupled to the pair of arms for allowing flexion and extension of the patient's knee and for increasing a medial/lateral distance between at least one arm of the pair of arms and the patient's knee during flexion and decreasing the medial/lateral distance during extension.

59. A knee brace for coupling to a patient's knee, the knee brace comprising:
a pair of arms for coupling to the patient's knee;
means coupled to the pair of arms for allowing flexion and extension of the patient's knee and for decreasing a medial/lateral distance between at least one arm of the pair of arms and the patient's knee during flexion and increasing the medial/lateral distance during extension.

60. A knee brace for coupling to a patient's knee, the knee brace comprising:
a first shell substantially conforming to the patient's thigh;
a second shell substantially conforming to the patient's calf;
a first arm coupled to the first shell;
a second arm coupled to the second shell;
a flexion/extension joint coupled between the first arm and the second arm; and
a first ramp positioned to engage the first arm and a second ramp positioned to engage the second arm, the first and second ramps shaped to move the first and second arms toward the patient's leg as the patient's knee extends and away from the patient's leg as the patient's knee flexes.

61. A knee brace for coupling to a patient's knee, the knee brace comprising:
a first shell substantially conforming to the patient's thigh;
a second shell substantially conforming to the patient's calf;
a first arm coupled to the first shell;
a second arm coupled to the second shell;
a flexion/extension joint coupled between the first arm and the second arm; and
a first ramp positioned to engage the first arm and a second ramp positioned to engage the second arm, the first and second ramps shaped to move the first and second arms toward the patient's leg as the patient's knee flexes and away from the patient's leg as the patient's knee extends.

62. A method of bracing a patient's knee, the method comprising:
securing a brace to the patient's knee;
allowing the patient's knee to flex and extend;
directing a first force toward a first point generally above the patient's knee;
directing a second force toward a second point generally below the patient's knee;
directing a third force generally away from the patient's knee;
increasing the first and second forces toward the patient's leg as the patient's knee extends; and
decreasing the first and second forces toward the patient's leg as the patient's knee flexes.

63. A method of bracing a patient's knee, the method comprising:
securing a brace to the patient's knee;
allowing the patient's knee to flex and extend;
directing a first force away from a first point generally above the patient's knee;
directing a second force away from a second point generally below the patient's knee;
directing a third force generally toward the patient's knee;
increasing the first and second forces away from the patient's leg as the patient's knee extends; and
decreasing the first and second forces away from the patient's leg as the patient's knee flexes.
